(19) United States
(12) Patent Application Publication
(10) Pub. No.: US 2023/0091164 A1
(43) Pub. Date: Mar. 23, 2023

(54) COCHLEAR IMPLANT ELECTRODE ARRAYS HAVING ORIENTATION INDICATORS AND COCHLEAR IMPLANTS INCLUDING THE SAME

(71) Applicants: Sara Elizabeth CLABEAUX, Ventura, CA (US); ADVANCED BIONICS AG, Staefa (CH)

(72) Inventor: Sarah Elizabeth Clabeaux, Ventura, CA (US)

(21) Appl. No.: 17/909,376

(22) PCT Filed: Mar. 31, 2020
(86) PCT No.: PCT/US2020/026049

§ 371 Date: Sep. 3, 2022

(51) Int. Cl. A61N 1/05; A61N 1/372; A61N 1/375

(52) U.S. Cl. CPC A61N 1/0541; A61N 1/37229; A61N 1/375

(57) ABSTRACT

A cochlear implant including a stimulation assembly including an antenna and a stimulation processor and a cochlear lead, operably connected to the stimulation processor, including an electrode array with a flexible body, a plurality of electrically conductive contacts on the flexible body, and a high contrast orientation indicator on a portion of the cochlear lead proximal of the electrode array.

### BACKGROUND

### 1. Field

The present disclosure relates generally to the implantable portion of implantable cochlear stimulation (or “ICS”) systems and, in particular, to electrode arrays.

### 2. Description of the Related Art

Referring to FIGS. 1 and 2, the cochlea 10 is a hollow, helically coiled, tubular bone (similar to a nautilus shell) that is divided into the scala vestibuli 12, the scala tympani 14 and the scala media 16 by the Reissner's membrane 18 and the basilar membrane 20. The cochlea 10, which typically includes approximately two and a half helical turns, is filled with a fluid that moves in response to the vibrations coming from the middle ear. As the fluid moves, a tectorial membrane 22 and thousands of hair cells 24 are set in motion. The hair cells 24 convert that motion to electrical signals that are communicated via neurotransmitters to the auditory nerve 26, and transformed into electrical impulses known as action potentials, which are propagated to structures in the brainstem for further processing. Many profoundly deaf people have sensorineural hearing loss that can arise from the absence or the destruction of the hair cells 24 in the cochlea 10. Other aspects of the cochlea 10 illustrated in FIGS. 1 and 2 include the medial wall 28, the lateral wall 30 and the modiolus 32.

ICS systems are used to help the profoundly deaf perceive a sensation of sound by directly exciting the intact auditory nerve with controlled impulses of electrical current. Ambient sound pressure waves are picked up by an externally worn microphone and converted to electrical signals. The electrical signals, in turn, are processed by a sound processor, converted to a pulse sequence having varying pulse widths, rates, and/or amplitudes, and transmitted to an implanted receiver circuit of the ICS system. The implanted receiver circuit is connected to an implantable lead with an electrode array that is inserted into the cochlea of the inner ear, and electrical stimulation current is applied to varying electrode combinations to create a perception of sound. The electrode array may, alternatively, be directly inserted into the cochlear nerve without residing in the cochlea. A representative ICS system is disclosed in U.S. Pat. No. 5,824,022, which is entitled “Cochlear Stimulation System Employing Behind-The-Ear Sound processor With Remote Control” and incorporated herein by reference in its entirety. Examples of commercially available ICS sound processors include, but are not limited to, the Advanced Bionics™ Harmony™ BTE sound processor, the Advanced Bionics™ Naida™ BTE sound processor and the Advanced Bionics™ Neptune™ body worn sound processor.

As alluded to above, some ICS systems include an implantable cochlear stimulator (or “cochlear implant”) having a lead with an electrode array, a sound processor unit (e.g., a body worn processor or behind-the-ear processor) that communicates with the cochlear implant, and a microphone that is part of, or is in communication with, the sound processor unit. The cochlear implant electrode array includes a flexible body formed from a resilient material and a plurality of electrically conductive contacts (e.g., sixteen platinum contacts) spaced along a surface of the flexible body. The contacts of the array are connected to lead wires that extend through the flexible body. Exemplary cochlear leads are illustrated in WO2018/031025A1 and WO2018/102695A1.

It is typically intended that after the electrode array is implanted within the cochlea, the contacts will all face the modiolus in the cochlea, which is where the spiral ganglion cells that innervate the hair cells are located. The perception of sound may be adversely impacted in those instances where some or all of the contacts in the electrode array are not facing the modiolus. The efficiency of the cochlear implant system is also adversely effected, e.g., battery life is reduced, when the contacts are not facing the modiolus because higher current may be required (as compared to a properly oriented electrode array) for the patient to perceive a particular level of loudness.

The present inventor has determined that conventional cochlear implant leads are susceptible to improvement. For example, electrode array insertion procedures are observed through a magnification device (e.g., a microscope, surgical loupes, an endoscope, or a video camera) focused on the round window under high magnification. This results in a shallow depth of focus and, accordingly, the inability to clearly see various portions of the lead that are proximal of the round window and in some instances indicative of lead orientation. Additionally, some recently proposed electrode array improvements have made it difficult for the surgeon to accurately identify the orientation of the electrode array relative to the modiolus. Relatively round atraumatic electrode array cross-sections as well as curved contacts that lack a flat light reflecting surface can make it difficult to discern the orientation of the lead.

### SUMMARY

A cochlear implant with a stimulation assembly in accordance with at least one of the present inventions includes an antenna and a stimulation processor and a cochlear lead, operably connected to the stimulation processor, including an electrode array with a flexible body, a plurality of electrically conductive contacts on the flexible body, and a high contrast orientation indicator on a portion of the cochlear lead proximal of the electrode array.

A method in accordance with at least one of the present inventions includes the steps of inserting a cochlear implant electrode array of a cochlear lead into a cochlea, observing the location of a high contrast orientation indicator on a portion of the cochlear lead proximal of the electrode array, determining, based on the location of the high contrast orientation indicator whether or not the electrode array is properly oriented, and rotating the electrode array in response to a determination that the electrode array is improperly oriented.

There are a number of advantages associated with such apparatus and methods. By way of example, but not limitation, the present apparatus and methods allow the surgeon to use a portion of the cochlear lead that is out of focus during the insertion procedure to determine whether or not the contacts are facing the modiolus.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of the exemplary embodiments will be made with reference to the accompanying drawings.

FIG. 1 is a section view of a cochlea.

FIG. 2 is another section view of the cochlea.

FIG. 3 is a plan view of a cochlear implant, including a stimulation assembly and a lead, in accordance with one embodiment of a present invention.

FIG. 4 is a bottom view of a portion of a cochlear implant lead illustrated in FIG. 3.

FIG. 5 is a perspective view of a portion of the cochlear implant lead illustrated in FIG. 3.

FIG. 6 is a section view taken along line 6-6 in FIG. 4.

FIG. 7 is a section view taken along line 7-7 in FIG. 4.

FIG. 8 is a perspective view of a portion of the cochlear implant lead illustrated in FIG. 3.

FIG. 9A is a perspective view of a portion of a cochlear implant lead in accordance with one embodiment of a present invention.

FIG. 9B is a section view of a portion of a cochlear implant lead in accordance with one embodiment of a present invention.

FIG. 10 is a side view of a portion of the cochlear implant lead illustrated in FIG. 3.

FIG. 11 is a perspective view of a portion of the cochlear implant lead illustrated in FIG. 3.

FIG. 12 is a view showing the implantation of a cochlear implant through the round window as observed through a magnification device.

FIG. 13 is a flow chart showing a method in accordance with one embodiment of a present invention.

FIG. 14 is a plan view of a cochlear implant, including a stimulation assembly and a lead, in accordance with one embodiment of a present invention.

FIG. 15 is a top view of a portion of a cochlear implant lead illustrated in FIG. 14.

FIG. 16 is a perspective view of a portion of a cochlear implant lead illustrated in FIG. 13.

FIG. 17 is an end view of a portion of a cochlear implant lead illustrated in FIG. 14.

FIG. 18 is a side view of a portion of a cochlear implant lead in accordance with one embodiment of a present invention.

FIG. 19 is a top view of a portion of a cochlear implant lead in accordance with one embodiment of a present invention.

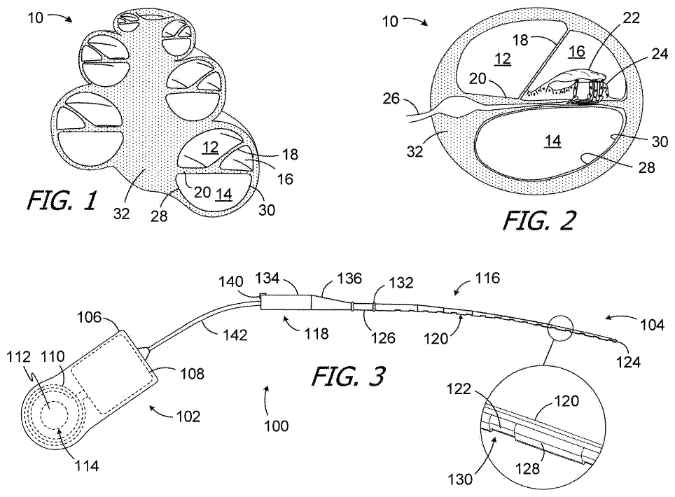

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

One example of a cochlear implant (or “implantable cochlear stimulator”) in accordance with at least some of the present inventions is illustrated in FIGS. 3-11. Referring first to FIG. 3, the exemplary cochlear implant 100 includes a stimulation assembly 102 and a cochlear lead 104.

A wide variety of stimulation assemblies may be combined with the present cochlear leads. The exemplary stimulation assembly 102 illustrated in FIG. 3 includes a flexible housing 106 formed from a silicone elastomer or other suitable material, a processor assembly 108, an antenna 110 that may be used to receive data and power by way of an external antenna that is associated with, for example, a sound processor unit, and a positioning magnet 112 located within a magnet pocket 114. The magnet 112 is used to maintain the position of a sound processor headpiece over the antenna 110. The cochlear implant may, in some instances, be configured in a manner that facilitates magnet removal and replacement. Here, the housing 106 may be provided with a magnet aperture (not shown) that extends from the magnet pocket 114 to the exterior of the housing.

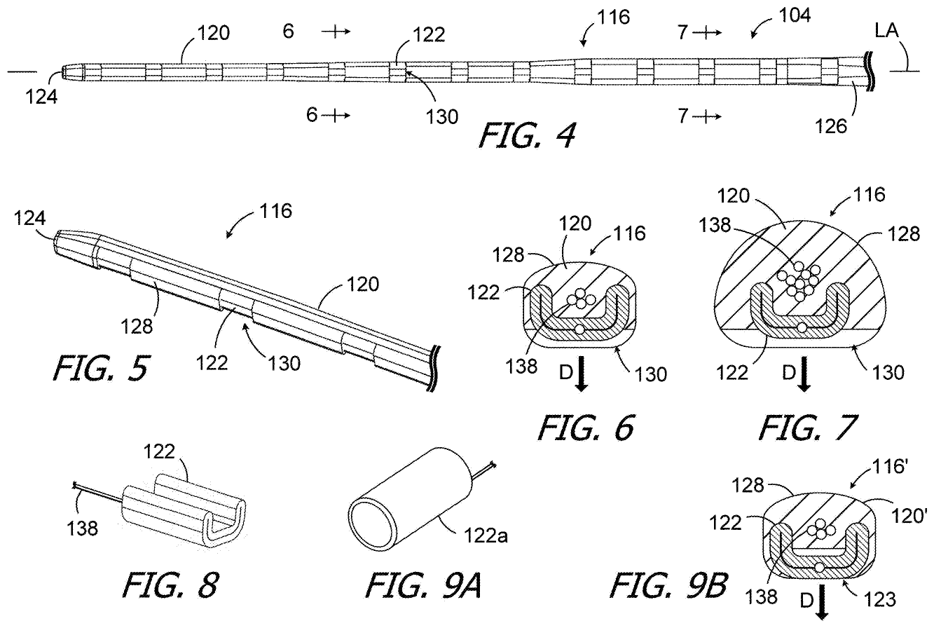

The exemplary cochlear lead 104 illustrated in FIGS. 3-11 includes an electrode array 116 and, in at least some instances, a wing 118 that functions as a handle for the surgeon during the implantation surgery. Wing 118 may comprise transparent or translucent colorless silicone rubber. Other types of handles may also be employed. The exemplary electrode array 116 has a flexible body 120 and a plurality of electrically conductive contacts 122 (e.g., sixteen contacts) spaced along the flexible body between the distal end 124 and the proximal end 126 of the flexible body. The electrically conductive contacts 122 (or “contacts”) may be located inward of the flexibly body outer surface 128 and exposed by way of a corresponding plurality of contact windows (or “windows”) 130 that extend through the outer surface of the flexible body to the contacts. [FIGS. 4-7.] The direction D that a contact 122 and a window 130 (when a window is employed) faces is the direction that the center of the exposed portion of the contact faces, as shown in FIGS. 6 and 7. In some instances, the proximal end 126 of the flexible body 120 may include one or more depth (or “cochleostomy”) markers 132 (FIG. 3) that are each a predetermined distance from the distal end 124. For example, the marker 132 closest to the wing 118 may represent insertion to 27 mm (ideal depth for a larger cochlea), while the other marker may represent insertion to 25 mm (ideal depth for a smaller cochlea).

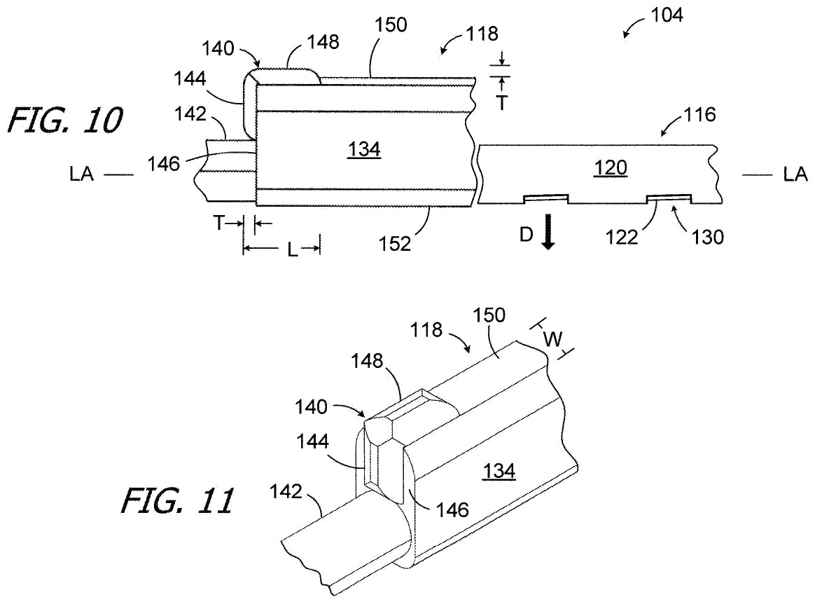

The wing 118 of the exemplary cochlear lead 104 illustrated in FIGS. 3-5 may include a rectangular portion 134 and a tapered portion 136 and, in addition to functioning as a handle, the wing provides tension relief for lead wires 138 (FIGS. 6-8) that do not run straight through the wing. The wing 118 also includes a high contrast orientation indicator 140, which is discussed in the greater detail below with reference to FIGS. 10-13. A tubular member 142, which may consist of tubes of different sizes, extends from the wing 118 to the stimulation assembly housing 106. The contacts 122 are connected to the lead wires 138, and the lead wires extend through the flexible body 120 and tubular member 142 to a connector (not shown) in the housing 106. The connection between the stimulation assembly 102 and a cochlear lead 104 may be a temporary connection, whereby the stimulation assembly and a cochlear lead may be disconnected from one another (e.g., for in situ replacement of the stimulation assembly), or a permanent connection.

Referring more specifically to FIGS. 4-7, and although the present inventions are not so limited, the flexible body 120 of the exemplary electrode array 116 has a non-circular shape with a flat bottom in a cross-section perpendicular to the longitudinal axis LA. The flexible body 120 may also be tapered, with a perimeter in a plane perpendicular to the longitudinal axis LA that is smaller at the distal end 124 than at the proximal end 126. The shape of the flexible body 120 also varies along the length of the flexible body. Any other suitable flexible body shapes (e.g., circular or oval), with or without a flat surface, may also be employed. Suitable materials for the flexible body 120 include, but are not limited to, electrically non-conductive resilient materials such as liquid silicone rubber (LSR), high temperature vulcanization (“HTV”) silicone rubbers, room temperature vulcanization (“RTV”) silicone rubbers, and thermoplastic elastomers (“TPEs”). These flexible body materials are typically transparent (or at least translucent) and colorless. Suitable materials for the contacts 122 include, but are not limited to, platinum, platinum-iridium, gold and palladium. Referring to FIG. 8, the exemplary contacts 122 may be generally U-shaped and may be formed by a placing a tubular workpiece into an appropriately shaped fixture, placing the end of a lead wire into the workpiece, and then applying heat and pressure to the workpiece to compress the workpiece onto the lead wire. The insulation may be removed from the portion of the lead wire within the workpiece prior to the application of heat and pressure or during the application of heat and pressure. Various examples of tubular workpieces being compressed onto lead wires are described in WO2018/031025A1 and WO2018/102695A1, which are incorporated herein by reference. The contact windows 130 extend from the outer surface 128 of the flexible body 120 to the contacts 122, thereby exposing portions of the contacts. In the exemplary implementation, the windows 130 are the same shape and expose the same portion of the associated contacts 122, as can be seen in FIGS. 6 and 7.

In other implementations, the contacts and/or windows in an electrode array may be different in sizes and/or shapes. For example, the contacts may be larger in the portion of the array that will be positioned in the basal region of the cochlea than the contacts in the portion that will be positioned in the apical region of the cochlea. The contacts may be rings 122a (FIG. 9A) that extend completely around the longitudinal axis LA for placement in the apical region. Alternatively, or in addition, the length (in the direction of the longitudinal axis LA) of the contacts in an electrode array may be the same or different. Referring to FIG. 9B, the position of the contacts 122 in electrode array 116′ is such that a portion 123 of each contact is aligned with the flexible body outer surface 128, thereby eliminating the need for a window.

High contrast orientation indicators in accordance with the present inventions may be placed on a portion of a cochlear lead that will be visible when the electrode array is being inserted into the cochlea. For example, high contrast orientation indicators in at least some implementations may be placed on a portion of the lead that is located proximal of the depth marker. High contrast orientation indicators in at least some implementations may also be placed on a portion of the lead that is located proximal of the electrode array. The high contrast orientation indicators in at least some implementations may be placed on a portion of the lead that is located at or proximal of the portion of the lead held by the surgeon during the insertion procedure. This portion of the lead may be 10 mm to 20 mm from the proximal-most contact that is connected to a lead wire and to which stimulation current is supplied. For example, a high contrast orientation indicator may be placed on the wing in at least some of the implementations described herein. In some instances, the high contrast orientation indicator on the wing will be 14 mm from the proximal-most contact that is connected to a lead wire and to which stimulation current is supplied.

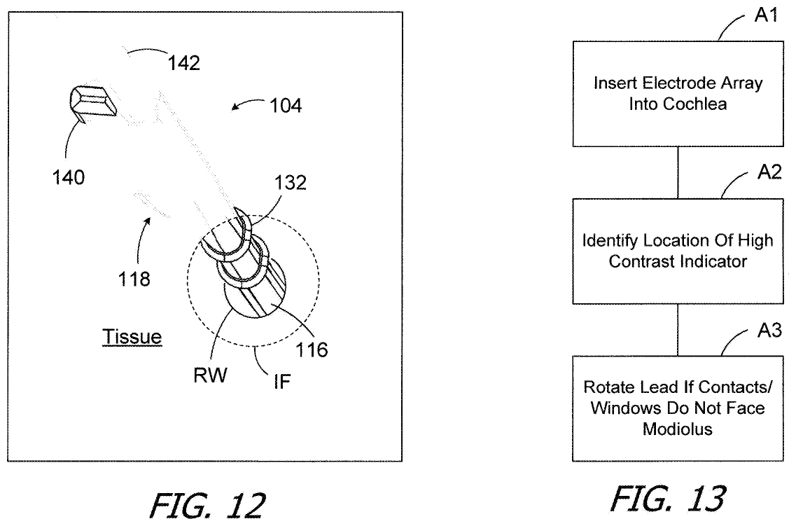

To that end, and referring to FIGS. 10 and 11, the exemplary high contrast orientation indicator 140 is in the form of a protrusion that includes a first portion 144 on the proximal end 146 of the wing 118 and a second portion 148 on the top side 150 of the wing. The high contrast orientation indicator 140 is, therefore, at or near the top side 150 of the wing 118 while the contacts 122 and windows 130 are associated with the bottom side 152 of the wing. The high contrast orientation indicator 140 is also associated with a portion of the wing 118 (i.e., the top side 150) that faces opposite the direction D that the contacts 122 and windows 130 face when the flexible body 120 is in a linear state. The linear state may be the relaxed state when the flexible body is not pre-curved or, in those instances where the flexible body is pre-curved, a straightened/stressed state. The high contrast orientation indicator 140 is also on a portion of the lead 104 that will be offset from the round window when the electrode array 116 is fully inserted into the cochlea. As a result, and as is discussed below with reference to FIGS. 12 and 13, the surgeon will be able to observe the location of the orientation indicator 140 and, based thereon, determine the orientation of the electrode array 116 relative to the modiolus.

The color of the high contrast orientation indicator 140 (as well as the other high contrast orientation indicators described herein) may be chosen to highly contrast with at least those portions of the cochlear lead 104 that are adjacent thereto. Alternatively, or in addition, the color of the high contrast orientation indicator 140 (as well as the other high contrast orientation indicators described herein) may be chosen to highly contrast with human tissue. Complementary colors, i.e., colors that when placed next to each other create the strongest contrast for those two colors, may be employed. Such colors are opposite one another on the color wheel.

For example, in those instances where the LSR or other material used to form the wing is translucent, the color of the associated high contrast orientation indicator may be a color that is complementary to red/pink human tissue near the cochlea, i.e., blue and/or green, to achieve for maximum contrast with the tissue. In those instances where the LSR or other material used to form the wing is not translucent, the respective colors of the wing and the high contrast orientation indicator may be complementary with one another and, at least somewhat complementary to the color of human tissue near the cochlea. In still other implementations, the colors of the wing and high contrast orientation indicator may be equidistant from red/pink human body tissue, and from one another, on the color wheel.

In the illustrated implementations, the markers 132 are the same color as the high contrast orientation indicator 140 (or the other high contrast orientation indicators described herein). In other implementations, the color of the markers 132 may be different than that of the high contrast orientation indicator 140 (or the other high contrast orientation indicators described herein).

In other implementations, black and white may be used to achieve high contrast. For example, the wing 118 (or other portion of the lead) may be white and the high contrast orientation indicator 140 (or the other high contrast orientation indicators described herein) may be black or blue or green. Alternatively, the wing 118 (or other portion of the lead) may be transparent or translucent and the high contrast orientation indicator 140 (or the other high contrast orientation indicators described herein) may be white. It should also be noted in this context that titanium dioxide, barium sulfate, and various bismuth compounds are commonly used to color silicone rubber white.

FIG. 12 is a magnification device view showing the electrode array 116 of the cochlear lead 104 being inserted into the round window RW. The portion of the image that is in focus (“IF”) is located within dashed line IF (which is shown merely for explanatory purposes). The remainder of the image, which is located outside of the dashed line IF, is out of focus. In the illustrated example, the proximal end of the electrode array 116, the round window RW, the markers 132 and a small portion of the wing 118 adjacent to the markers are in focus. The remainder of the cochlear lead 104 is out of focus. Nevertheless, due to the high contrast between the wing 118 and the high contrast orientation indicator 140, the orientation indicator can be readily identified despite being out of focus. Given the relative positions of the exposed portions of the contacts 122 (as well as the positions of the contact windows 130 when present) and the high contrast indicator 140 on the exemplary cochlear lead 104, the surgeon will be able to discern whether or not the contacts 122 and contact windows 130 are facing the modiolus, i.e., whether or not the direction D is pointing at the modiolus. In the illustrated implementation, where the contacts 122 and contact windows 130 are associated with the side of the electrode array 116 that is offset 180 degrees around the longitudinal axis LA (FIG. 10) from the portion 148 of the indicator 140 on the top side 150 of the wing 118, orienting the cochlear lead 104 in such a manner that the indicator portion 148 faces in the inferior direction (i.e., is pointed in the head-to-feet direction) will result in the contacts 122 and contact windows 130 facing in the superior direction (i.e., is pointed in the feet-to-head direction) and facing the modiolus and the direction D pointing at the modiolus. The cochlear lead 104 may be rotated (or pulled in the proximal direction and rotated) in those instances where the location of the high contrast orientation indicator 140 is indicative of contacts 122 that do not face the modiolus. Alternatively, in those instances where the location of the high contrast orientation indicator 140 is indicative of contacts 122 that face the modiolus, the lead 104 will not be rotated.

The method steps described with reference to FIG. 12 may be summarized in the manner illustrated in FIG. 13. First, the electrode array 116 of the cochlear implant lead 104 may be advanced into the cochlea by way of the round window or other suitable access point (step A1). Next, in step A2, the surgeon will identify the location of the high contrast orientation indicator 140, which may be in focus or out of focus, by way of a microscope or other suitable magnification device. The location of the high contrast orientation indicator 140 is, as described above, indicative of the orientation of the contacts 122 and contact windows 130 relative to the modiolus. Should it be determined that the orientation of the electrode array 116 is improper, the array may be rotated or otherwise adjusted. Alternatively, should it be determined that the orientation of the electrode array 116 is proper, the surgical procedure may simply proceed. It should be noted that the high contrast orientation indicators described herein may be used by surgeons in any suitable manner to ascertain the orientation of the contacts 122 and contact windows 130 relative to the modiolus. For example, the surgeon may use an anatomic feature such as the inferior-superior direction of the patient's body. The round window is another anatomic feature that may be used, i.e., the location of the high contrast orientation indicator relative to the round window may be used by the surgeon to determine contact orientation relative to the modiolus.

A variety of techniques may be used to color the high contrast orientation indicator 140. For example, in those instances where the wing 118 and indicator 140 are molded simultaneously, a coating of dye or other suitable high contrast material may be applied to the indicator 140. In other instances, appropriately colored silicone (or other suitable material) may be overmolded onto the wing 118 to form the indicator. Alternatively, a piece of appropriately colored metal wire, silicone rubber, plastic, or fiber (e.g., woven polyethylene fiber) may be insert molded into the wing 118 to form the indicator. The high contrast material could also be radiopaque in order to allow the orientation of the associated electrode array to be assessed during post-operative scans.

With respect to the dimensions, the high contrast orientation indicator 140 should be relatively small and in a location on the associated lead that increases the likelihood that the indicator will be seen and decreases the likelihood that the indicator will obstruct the surgeon's magnified view of the round window. In the illustrated embodiment, and referring to FIGS. 10 and 11, the thickness T of the exemplary high contrast orientation indicator 140 is about 0.1 mm to 0.2 mm, the width W is about 0.5 mm to 0.8 mm wide, and the length L of each of the first and second portions 144 and 148 is about 0.8 mm to 1.0 mm.

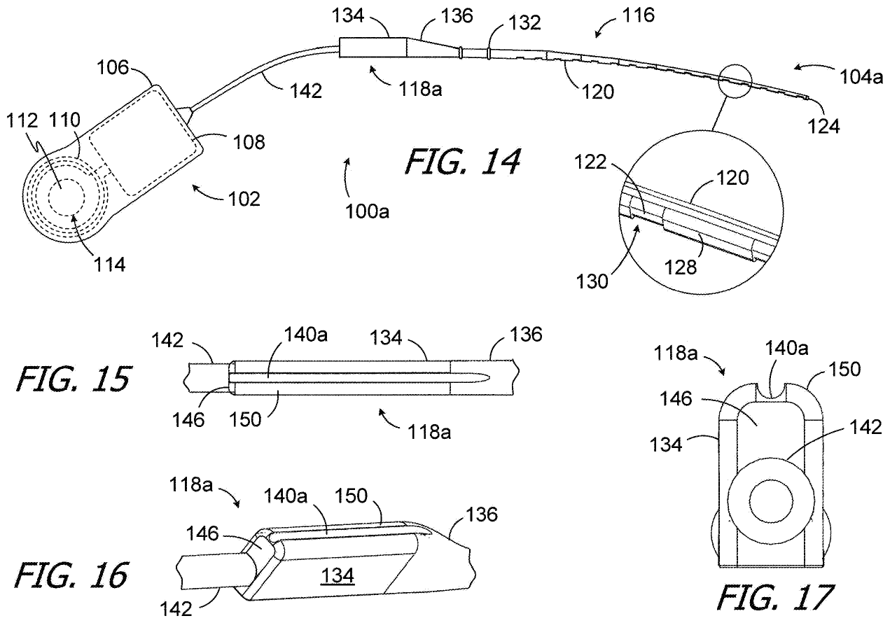

Another exemplary cochlear implant is generally represented by reference numeral 100a in FIG. 14. The cochlear implant 100a is substantially similar to cochlear implant 100 and similar elements are represented by similar reference numerals. Here, however, the cochlear lead 104a includes a wing 118a with a high contrast orientation indicator 140a in the form of an indentation in the top side 150 of the wing, as is illustrated in FIGS. 15-17. The high contrast orientation indicator 140a extends from the proximal end 146 of the wing 118a, along the entire length of the rectangular portion 134, and along part of the tapered portion 136. The use of an elongate indentation, as opposed to an elongate protrusion, eliminates the possibility the high contrast orientation indicator will obstruct the view of the round window or other anatomic structure. In still other implementations, the high contrast orientation indicator may be flat (e.g., a stripe).

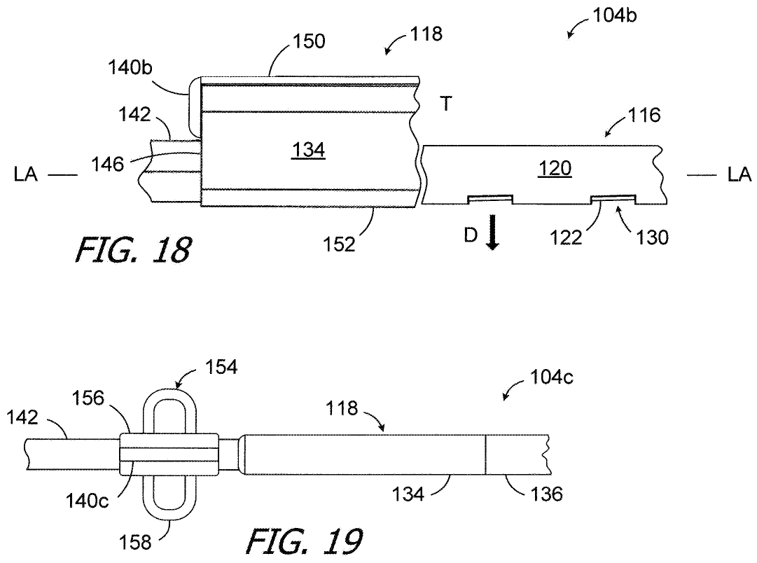

The exemplary cochlear implant lead generally represented by reference numeral 104b in FIG. 18 is substantially similar to cochlear implant lead 104 and similar elements are represented by similar reference numerals. Here, however, the high contrast orientation indicator 140b is located only on the proximal end 146 of the wing 118.

High contrast orientation indicators may also be associated with other aspects of cochlear leads. To that end, and turning to FIG. 19, the exemplary cochlear lead 104c is substantially similar to lead 104 and similar elements are represented by similar reference numerals. The cochlear lead 104c may be connected to and used in conjunction with, for example, the stimulation assembly 102 (FIG. 3). Here, however, the cochlear lead 104c is provided with an anchor 154 that is mounted on the tubular member 142 in such a manner that the anchor is not readily rotatable relative to the tubular member. The anchor can be used to stabilize the electrode array by attaching it to tissue with a suture, staple, clip, or the like. The anchor 154 includes a sheath 156 that is secured to the tubular member 142, a pair of fixation tabs 158 that extend outwardly from the sheath, and a high contrast orientation indicator 140c in the form of an indentation in the sheath (as shown) or in the form of a protrusion. The orientation of the indicator 140c is fixed relative to the electrode array and, in the illustrated implementation, is the same as that of the indicators 140 and 140a. The axial position and radial orientation of the sheath 156 may be fixed or semi-fixed to the tubular member 142 through the use of techniques such as compression fitting and over-molding. Alternatively, the radial orientation of the sheath 156 may be fixed with respect to the tubular member 142, such as by using mating tracks (not shown), while the axial position may be adjustable. The wing 118 does not include a high contrast orientation indicator. In other implementations, both the wing and the anchor may be provided with respective high contrast orientation indicators.

Although the inventions disclosed herein have been described in terms of the preferred embodiments above, numerous modifications and/or additions to the above-described preferred embodiments would be readily apparent to one skilled in the art. By way of example, but not limitation, the present high contrast orientation indicators need not be on or part of the outer surface of the associated device. Instead, high contrast orientation indicators may in some instances be located under the surface of a transparent (or at least translucent) structure or coated with a transparent (or at least translucent) material. The inventions also include any combination of the elements from the various species and embodiments disclosed in the specification that are not already described. It is intended that the scope of the present inventions extend to all such modifications and/or additions and that the scope of the present inventions is limited solely by the claims set forth below.

1. A cochlear implant, comprising:
a stimulation assembly including an antenna and a stimulation processor operably connected to the antenna; and
a cochlear lead, operably connected to the stimulation processor, including an electrode array with a flexible body, a plurality of electrically conductive contacts on the flexible body with one of the electrically conductive contacts defining a proximal-most electrically conductive contact, a high contrast orientation indicator on a portion of the cochlear lead proximal of the electrode array, and a depth marker located between the proximal-most electrically conductive contact and the high contrast orientation indicator.

2. (canceled)

3. A cochlear implant as claimed in claim 1, wherein
the cochlear lead defines a longitudinal axis;
the electrically conductive contacts face in a first direction relative to the longitudinal axis; and
at least a portion of the high contrast orientation indicator faces in a second direction that is opposite the first direction.

4. A cochlear implant as claimed in claim 1, wherein
the cochlear lead includes a handle portion; and
the high contrast orientation indicator is on the handle portion.

5. A cochlear implant, comprising:
a stimulation assembly including an antenna and a stimulation processor operably connected to the antenna; and
a cochlear lead, operably connected to the stimulation processor, including an electrode array with a flexible body, a plurality of electrically conductive contacts on the flexible body, a wing proximal of the electrode array, and a high contrast orientation indicator on the wing.

6. A cochlear implant as claimed in claim 5, wherein
the wing includes a proximal end and a top side; and
the high contrast orientation indicator includes a first portion on the wing proximal end and a second portion on the wing top side.

7. A cochlear implant as claimed in claim 5, wherein
the wing includes a proximal end and a top side; and
the high contrast orientation indicator is on the wing proximal end.

8. A cochlear implant as claimed in claim 1, wherein
the cochlear lead includes an anchor; and
the high contrast orientation indicator is on the anchor.

9. A cochlear implant as claimed in claim 1, wherein
the high contrast orientation indicator comprises a protrusion.

10. A cochlear implant as claimed in claim 1, wherein
the high contrast orientation indicator comprises an indentation.

11. A cochlear implant as claimed in claim 1, wherein
the portion of the cochlear lead on which the high contrast orientation indicator is located is translucent; and
the high contrast orientation indicator is blue or the high contrast orientation indicator is green.

12. A cochlear implant as claimed in claim 1, wherein
the portion of the cochlear lead on which the high contrast orientation indicator is located is a first color; and
the high contrast orientation indicator is a second color that is complementary with the first color.

13. A cochlear implant as claimed in claim 1, wherein
the portion of the cochlear lead on which the high contrast orientation indicator is located is a first color;
the high contrast orientation indicator is a second color; and
the first and second colors are equidistant from human body tissue color and from another on the color wheel.

14. A cochlear implant as claimed in claim 1, wherein
the plurality of electrically conductive contacts includes a proximal-most electrically conductive contact; and
the high contrast orientation indicator is at least 10 mm from the proximal-most electrically conductive contact that is connected to a lead wire and to which stimulation current is supplied.

15. A cochlear implant as claimed in claim 1, wherein
the plurality of electrically conductive contacts includes a proximal-most electrically conductive contact; and
the high contrast orientation indicator is 10 mm to 20 mm from the proximal-most electrically conductive contact that is connected to a lead wire and to which stimulation current is supplied.

16. A cochlear implant as claimed in claim 1, wherein
the high contrast orientation indicator is black or white.

17. A method, comprising the steps of:
inserting a cochlear implant electrode array of a cochlear lead into a cochlea;
observing the location of a high contrast orientation indicator on a portion of the cochlear lead proximal of the electrode array;
determining, based on the location of the high contrast orientation indicator whether or not the electrode array is properly oriented; and
rotating the electrode array in response to a determination that the electrode array is improperly oriented.

18. A method as claimed in claim 17, wherein
observing the location of the high contrast orientation indicator comprises observing the high contrast orientation indicator through a magnification device while the high contrast orientation indicator is out of focus.

19. A method as claimed in claim 17, wherein
the cochlear lead is part of the cochlear implant claimed in claim 1.

20. A method as claimed in claim 17, wherein
the cochlear lead is part of the cochlear implant claimed in claim 5.
